(19) United States
(12) Patent Application Publication
(10) Pub. No.: US 2017/0316562 A1
(43) Pub. Date: Nov. 2, 2017

(54) DETERMINING AT LEAST ONE PROTOCOL PARAMETER FOR A CONTRAST AGENT-ASSISTED IMAGING METHOD

(71) Applicant: Siemens Healthcare GmbH, Erlangen (DE)

(72) Inventors: Ulrike HABERLAND, Erlangen (DE); Andreas WIMMER, Forchheim (DE)

(73) Assignee: Siemens Healthcare GmbH, Erlangen (DE)

(21) Appl. No.: 15/492,032

(22) Filed: Apr. 20, 2017

(30) Foreign Application Priority Data
Apr. 28, 2016 (DE) 102016207291.9

(51) Int. Cl. G06T 7/00; A61B 6/00; G06T 7/13; A61B 5/055; A61B 6/03

(52) U.S. Cl. CPC G06T 7/0012; A61B 6/032; A61B 6/5217; A61B 6/481; G06T 7/97; A61B 5/055; G06T 7/13; G06T 2207/10081

(57) ABSTRACT

A method for determining at least one protocol parameter for a contrast agent-assisted acquisition of images of a region that is to be examined of an examination subject via a medical imaging device is described. In an embodiment of the method, an acquisition of external images of the exterior of the examination subject is first performed with the aid of an external image acquisition unit. At least one body dimension of the examination subject is then determined on the basis of the acquired external images. Finally, at least one contrast agent protocol parameter is determined on the basis of the at least one determined body dimension. Furthermore, an image acquisition parameter determination device is described. In addition, an imaging medical device is also described.

### PRIORITY STATEMENT

The present application hereby claims priority under 35 U.S.C. §119 to German patent application number DE 102016207291.9 filed Apr. 28, 2016, the entire contents of which are hereby incorporated herein by reference.

### FIELD

At least one embodiment of the invention generally relates to a method for automatically determining at least one protocol parameter for a contrast agent-assisted acquisition of images of a region that is to be examined of an examination subject. At least one embodiment of the invention also generally relates to an image acquisition parameter determination device. Finally, At least one embodiment of the invention generally relates to an imaging medical device.

### BACKGROUND

State-of-the-art imaging methods are often enlisted as an aid to generating two- or three-dimensional image data which may be used for visualizing an imaged examination subject as well as for further applications besides.

The imaging methods are frequently based on the detection of X-ray radiation, with data referred to as projection measurement data being generated in the process. For example, projection measurement data can be acquired with the aid of a computed tomography system (CT system). In CT systems, a combination consisting of X-ray source and oppositely positioned X-ray detector is arranged on a gantry and typically rotates around a measurement chamber in which the examination subject (referred to in the following without loss of generality as the patient) is situated. In this case the center of rotation (also known as the “isocenter”) coincides with an axis referred to as system axis z. In the course of one or more revolutions, the patient is irradiated with X-ray radiation of the X-ray source, during which process projection measurement data or X-ray projection data is acquired with the aid of the oppositely disposed X-ray detector.

The X-ray detectors used in CT imaging generally comprise a plurality of detection units, which in most cases are arranged in the form of a regular pixel array. Each of the detection units generates a detection signal for X-ray radiation that is incident on the detection units, which detection signal is analyzed in terms of intensity and spectral distribution of the X-ray radiation at specific time instants in order to obtain inferences in relation to the examination subject and to generate projection measurement data.

Other imaging techniques are based on magnetic resonance tomography, for example. During the generation of magnetic resonance images, the body that is to be examined is exposed to a relatively high basic magnetic field, for example of 1.5 tesla, 3 tesla, or, in the case of more recent high magnetic field systems, even 7 tesla. A radiofrequency excitation signal is then transmitted via a suitable antenna device, causing the nuclear spins of specific atoms excited into resonance by way of the radiofrequency field in the given magnetic field to be tipped through a defined flip angle with respect to the magnetic field lines of the basic magnetic field. The radiofrequency signal emitted during the relaxation of the nuclear spins, known as the magnetic resonance signal, is then intercepted via suitable antenna devices, which may also be identical to the transmit antenna device. Finally, the raw data acquired in this way is used in order to reconstruct the desired image data. While the radiofrequency signals are being sent and read out or received, defined magnetic field gradients are superimposed in each case on the basic magnetic field for spatial encoding purposes.

In the imaging of structures of the body of patients by way of the imaging methods that have been briefly outlined, substances known as contrast agents are often used in addition. An important protocol parameter when performing a contrast agent-assisted medical imaging procedure relates to the volume of contrast agent that is required for the image acquisition process. This is determined for instance on the basis of the weight of the patient. However, details provided by patients are often inaccurate, which means it is necessary beforehand to take a weight measurement with the aid of a weighing machine.

A further protocol parameter for a contrast agent-assisted medical imaging procedure relates to the point in time at which the contrast agent, after having been injected into the body of the patient, is present in that region of the patient's body that is to be examined and at which the imaging can be started. This time parameter is also referred to as the delay time.

Often, the start time for the imaging is simply estimated on the basis of empirical values. Such an approach is not particularly precise, however. It may happen as a consequence that the time at which the image acquisition procedure is started is set too late, with the result that the contrast agent has already passed through the region to be examined and yields no benefit. It may possibly be necessary in this situation to administer more contrast agent so that at least a portion thereof is still present in the region to be examined during the acquisition time, although this entails an additional exposure for the patient. In principle, it is aimed to achieve the shortest possible residence time of the contrast agent in the body, because the contrast agent can have debilitating side-effects on the human body. If the image acquisition procedure is started too early, the contrast agent will not yet be present in the region to be examined at the time of image acquisition, which is associated with a deterioration in contrast or can result in a degradation in the image quality. In the worst case it may even be necessary to repeat the image acquisition procedure as well as the administration of contrast agent, which likewise constitutes an additional exposure for the patient.

One possibility of making the contrast agent visible in the body prior to the actual imaging consists in carrying out a procedure known as a bolus tracking scan (BT scan for short), which is performed prior to the actual imaging. Such a BT scan can be a time-dependent image acquisition procedure, for example a CT scan, conducted at a low resolution, by which a time-density curve of a subregion of a region to be examined is acquired.

Typically, such a subregion for a BT scan comprises a slice which is embodied and also considered as orthogonal to the z-direction, i.e. the direction of the system axis of the imaging system. However, data can also be acquired at different levels, in particular in magnetic resonance tomography. In real-world practice, during the performance of the BT scan, attenuation values are acquired as a function of time and space in a subregion of the region to be examined, in which subregion an artery is present in most cases. If the injected contrast agent now flows through the observed artery, the attenuation values are increased significantly. If a predetermined threshold value for the attenuation values is exceeded, for example 150 Hounsfield units (HU), this can be interpreted as proof that the contrast agent is present in sufficient concentration in the region to be examined, and the actual image acquisition can be started.

However, such a bolus tracking scan performed in advance is time-consuming and in the case of an MRT or CT acquisition procedure also signifies an additional exposure of the patient to be examined due to radiation or energy input.

### SUMMARY

An embodiment of the present invention discloses, in connection with the contrast agent-assisted imaging, a more user-friendly and nonetheless precise method for determining at least one protocol parameter for the imaging.

Embodiments of the invention are directed to a method for determining at least one protocol parameter for a contrast agent-assisted acquisition of images; an image acquisition parameter determination device; and an imaging medical device.

In at least one embodiment, the inventive method is for determining at least one protocol parameter for a contrast agent-assisted acquisition of images of a region to be examined of an examination subject via a medical imaging device. An acquisition of external images of externally visible features of the examination subject is performed beforehand with the aid of an external image acquisition unit. The external image acquisition unit serves to perform an acquisition of external images of the examination subject with minimum use of resources in the shortest possible time and with the lowest possible exposure for the patient. Whereas the actual medical imaging device is intended to acquire images of the interior of the examination subject, the acquisition of external images via the additional external image acquisition unit is limited to the acquisition of images of the externally visible features or, as the case may be, of the surface and the contours of the examination subject.

An embodiment of the inventive image acquisition parameter determination device serves for determining at least one protocol parameter of a contrast agent-assisted acquisition of images of a region to be examined of an examination subject via a medical imaging device. For that purpose, an embodiment of the inventive image acquisition parameter determination device of at least one embodiment comprises an additional external image acquisition unit for performing an acquisition of external images of external features of the examination subject. Also part of an embodiment of the inventive image acquisition parameter determination device is a body dimension determination device for determining at least one body dimension of the examination subject on the basis of the acquired external images. In addition, an embodiment of the inventive image acquisition parameter determination device comprises a contrast agent protocol parameter determination unit which is configured to determine contrast agent protocol parameters on the basis of the at least one determined body dimension.

An embodiment of the inventive imaging medical device, preferably a computed tomography system, comprises a scan unit for scanning a region to be examined of an examination subject. It furthermore comprises a control unit for controlling the scan unit. In addition, an embodiment of the inventive imaging medical device comprises an image acquisition parameter determination device.

The majority of the main components of an embodiment of the inventive image acquisition parameter determination device can be embodied in the form of software components. This relates in particular to the body dimension determination device and the contrast agent-protocol parameter determination unit. Basically, however, some of these components can also be realized in the form of software-assisted hardware, for example FPGAs or the like, in particular when there is a requirement for particularly fast calculations. Equally, the required interfaces can be embodied as software interfaces, for example when it is simply a matter of importing data from other software components. They can, however, also be embodied as hardware-based interfaces which are controlled by suitable software.

A largely software-based implementation has the advantage that control devices already used previously in the prior art can also be easily upgraded by way of a software update in order to operate in the manner according to the invention. In that respect, in an embodiment, a corresponding computer program product includes a computer program which can be loaded directly into a memory device of a control device of an imaging system, preferably a computed tomography system, and having program sections for the purpose of carrying out all steps of the method according to an embodiment of the invention when the program is executed in the control device.

A computer-readable medium, for example a memory stick, a hard disk or some other transportable or permanently installed data carrier, on which the program sections of the computer program that can be read in and executed by a computer unit of the control device are stored, may be used for transporting the computer program to the control device and/or for storing the same on or in the control device. For this purpose, the computer unit may have e.g. one or more cooperating microprocessors or the like.

In a special embodiment of the method according to an embodiment of the invention, a virtual model of the examination subject is used for particularly precise determination of the at least one body dimension of the examination subject, which virtual model is fitted to the data obtained via the external image acquisition procedure. If the examination subject is a human being, such a virtual model is commonly referred to as an avatar.

### BRIEF DESCRIPTION OF THE DRAWINGS

The invention is explained once again in more detail hereinbelow on the basis of example embodiments and with reference to the attached figures, in which:

FIG. 1 shows a flowchart which illustrates a method for determining at least one protocol parameter for a contrast agent-assisted acquisition of images of a region that is to be examined of an examination subject via a medical imaging device,

FIG. 2 shows a block diagram which illustrates an image acquisition parameter determination device according to an example embodiment of the invention, and

FIG. 3 shows a computed tomography system according to an example embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5,Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In at least one embodiment, the inventive method is for determining at least one protocol parameter for a contrast agent-assisted acquisition of images of a region to be examined of an examination subject via a medical imaging device. An acquisition of external images of externally visible features of the examination subject is performed beforehand with the aid of an external image acquisition unit. The external image acquisition unit serves to perform an acquisition of external images of the examination subject with minimum use of resources in the shortest possible time and with the lowest possible exposure for the patient. Whereas the actual medical imaging device is intended to acquire images of the interior of the examination subject, the acquisition of external images via the additional external image acquisition unit is limited to the acquisition of images of the externally visible features or, as the case may be, of the surface and the contours of the examination subject.

In the simplest case, the acquired external image can be a two-dimensional image, referred to in the following as a 2D image, which is represented in monochrome or in color.

At least one body dimension of the examination subject is determined, preferably automated, on the basis of the acquired external image. Finally, at least one contrast agent protocol parameter is determined on the basis of the at least one determined body dimension. The contrast agent protocol is therefore individually adapted in advance with the aid of the at least one dimension of the examination subject determined on the basis of the externally acquired image so that an acquisition of images of the internal structures of the examination subject is achieved with improved image quality with the aid of the following contrast agent-assisted imaging. The steps for determining the contrast agent protocol parameters are preferably performed in an automated manner in order to reduce the workload of operating staff.

In contrast to conventional methods, no additional internal images acquired by the medical imaging device itself are required in order to estimate the contrast agent protocol parameters, so that an additional exposure of the patients occurring for example in the case of CT imaging methods is avoided.

Furthermore, the external image acquisition procedure can also be performed in a more time-saving manner with the aid of the medical imaging device compared to a more complicated survey scan, with the result that the total patient examination time can be reduced and patient comfort increased. The shorter examination time also enables a higher throughput of the medical imaging device to be achieved, thus improving the operational cost-effectiveness of the device. Furthermore, the automated determination of patient parameters is more accurate or more consistent than, for example, details given by the patients.

What is to be understood in this context by a contrast agent-assisted imaging method or a contrast agent-assisted acquisition of images generated therewith are all types of imaging methods imaging internal structures of an examination subject in which a contrast agent is additionally used in order to enhance the contrast of structures that are to be imaged. Examples of this are contrast agent-assisted CT imaging methods and contrast agent-assisted MR imaging methods for visualizing blood vessels, internal organs or parts of organs. Furthermore, they also include procedures referred to as perfusion measurements in which the blood flow through organs is visualized and examined.

An embodiment of the inventive image acquisition parameter determination device serves for determining at least one protocol parameter of a contrast agent-assisted acquisition of images of a region to be examined of an examination subject via a medical imaging device. For that purpose, an embodiment of the inventive image acquisition parameter determination device of at least one embodiment comprises an additional external image acquisition unit for performing an acquisition of external images of external features of the examination subject. Also part of an embodiment of the inventive image acquisition parameter determination device is a body dimension determination device for determining at least one body dimension of the examination subject on the basis of the acquired external images. In addition, an embodiment of the inventive image acquisition parameter determination device comprises a contrast agent protocol parameter determination unit which is configured to determine contrast agent protocol parameters on the basis of the at least one determined body dimension.

An embodiment of the inventive imaging medical device, preferably a computed tomography system, comprises a scan unit for scanning a region to be examined of an examination subject. It furthermore comprises a control unit for controlling the scan unit. In addition, an embodiment of the inventive imaging medical device comprises an image acquisition parameter determination device.

The implementation of an embodiment of the invention in a CT system has the advantage that the duration of a scan performed by a CT system is relatively short. It amounts to only a few seconds, compared to the acquisition of images via MRT systems, which may require several minutes. This is particularly advantageous when it comes to the examination of emergency patients, in which any delay may be life-threatening. Furthermore, CT systems are more widely established and less expensive than MRT systems.

On the other hand, MRT systems have the advantage that an examination carried out using them involves no exposure to X-ray radiation and the soft tissue contrast in an image acquired using an MR system is improved in comparison with a CT system.

The majority of the main components of an embodiment of the inventive image acquisition parameter determination device can be embodied in the form of software components. This relates in particular to the body dimension determination device and the contrast agent-protocol parameter determination unit. Basically, however, some of these components can also be realized in the form of software-assisted hardware, for example FPGAs or the like, in particular when there is a requirement for particularly fast calculations. Equally, the required interfaces can be embodied as software interfaces, for example when it is simply a matter of importing data from other software components. They can, however, also be embodied as hardware-based interfaces which are controlled by suitable software.

A largely software-based implementation has the advantage that control devices already used previously in the prior art can also be easily upgraded by way of a software update in order to operate in the manner according to the invention. In that respect, in an embodiment, a corresponding computer program product includes a computer program which can be loaded directly into a memory device of a control device of an imaging system, preferably a computed tomography system, and having program sections for the purpose of carrying out all steps of the method according to an embodiment of the invention when the program is executed in the control device.

In particular, the computer program product may be the computer program or comprise at least one additional component as well as the computer program. The at least one additional component of the computer program product may be chosen for example from the group consisting of
a memory device on which at least a part of the computer program is stored,
a key for authenticating a user of the computer program, wherein the key may be embodied in the form of hardware (e.g. a dongle) and/or software,
documentation relating to the computer program, in a printed and/or digital version,
a first additional computer program which forms a software package in combination with the computer program,
a second additional computer program which is embodied for compressing and/or decompressing the computer program and/or which forms an installation package in combination with the computer program,
a third additional computer program which is embodied for distributing processing steps that are carried out during the execution of the computer program to different processing units of a cloud computing system and/or which, together with the computer program, forms a cloud computing application, and combinations thereof.

A computer-readable medium, for example a memory stick, a hard disk or some other transportable or permanently installed data carrier, on which the program sections of the computer program that can be read in and executed by a computer unit of the control device are stored, may be used for transporting the computer program to the control device and/or for storing the same on or in the control device. For this purpose, the computer unit may have e.g. one or more cooperating microprocessors or the like.

The claims as well as the following description in each case contain particularly advantageous embodiments and developments of the invention. In this regard, in particular the claims of one claims category may also be developed analogously to the dependent claims of a different claims category. Furthermore, the various features of different example embodiments and claims may also be combined within the scope of the invention in order to create new example embodiments.

In an embodiment of the inventive method for determining at least one protocol parameter for a contrast agent-assisted acquisition of images, the at least one determined contrast agent protocol parameter comprises one or more of the following variables:
a required contrast agent volume,
a start time for the contrast agent-assisted image acquisition procedure.

An embodiment variant of the invention provides that the at least one determined contrast agent protocol parameter is the required volume of contrast agent. The required contrast agent volume can be determined for example on the basis of the at least one determined body dimension.

An embodiment variant of the invention provides that the at least one determined contrast agent protocol parameter is the start time of the contrast agent-assisted image acquisition procedure. The start time of the contrast agent-assisted image acquisition procedure can be determined for example on the basis of the at least one determined body dimension.

To put it more precisely, a minimum period of time required by a contrast agent to arrive in an examination region can be determined with the aid of the method. In this way an improvement in the determining of a favorable start time for a contrast agent-assisted acquisition of images is achieved.

As already mentioned, a certain minimum amount of contrast agent is required in order to achieve an optimal image contrast in an image acquisition procedure performed via the imaging medical device in question. With the aid of the determined dimensions of the examination subject it is now possible to determine an optimal contrast agent volume which satisfies the desired requirements in terms of image contrast and at the same time does not exceed a reasonable maximum value. If the examination subject is a patient, for example an animal or a human being, then it is for example additionally of interest that the contrast agent volume administered to the patient does not exceed a maximum value in order not to subject the patient to an undue exposure.

As likewise already explained, the start time of a contrast agent-assisted image acquisition procedure must be synchronized with the time of arrival of the administered contrast agent in the examination region of the examination subject in order to achieve an optimal effect of the contrast agent in terms of an enhanced image contrast. The length of the transportation path of the contrast agent used can be deduced from the determined body dimensions. If the flow rate of the contrast agent through the examination subject is known, the time of arrival of the contrast agent in the desired examination region can be calculated on the basis of the determined length of the transportation path and the flow rate. With the determined distance between body regions it is therefore possible to make an improved prediction in terms of a likely delay time in the case of the injection of a contrast agent. For example, a minimum delay time with which the contrast agent reaches the target region can be calculated by way of the measured distance between the injection site, the heart and the target organ for the imaging. At the same time, further parameters, such as the behavior of the circulatory system and the vessel diameter, for example, can either be estimated on the basis of statistics or also be adjusted as appropriate if the individual values are known.

In order to determine the required contrast agent volume, the weight of the examination subject can be determined initially for example on the basis of the at least one body dimension determined with the aid of the acquired external images. If, for example, a specific weight of the type of the examination subject is known, then it is possible for example firstly to determine the volume of the examination subject on the basis of the latter's external dimensions and the total weight thereof from the volume and the specific weight. Alternatively, a table in which distance values or volume values are assigned to a specific patient weight may also be used in order to determine the weight of a patient.

Next, the required contrast agent volume is determined on the basis of the determined weight of the examination subject. Generally, the required contrast agent volume behaves approximately proportionally to the weight of the examination subject, so that knowing the weight of the examination subject as well as, for example, a reference value for the required contrast agent volume for a reference weight is sufficient in order to estimate a required contrast agent volume for an arbitrary weight. In addition, the required contrast agent volume is also dependent on the diameter of the region perfused with the contrast agent, since a stronger attenuation or a stronger contrast is achieved by way of the contrast agent in a narrow diameter than in a greater diameter. To determine the contrast agent volume more precisely, the patient diameter can be determined for example by way of a topogram or the patient contours. It may also be determined with the aid of an avatar. After the contrast agent volume has been determined, a finalized injection protocol can be specified for a scan protocol and transmitted to the injector, i.e. the apparatus for injecting the contrast agent, comprising a control device for controlling the administration of the contrast agent.

In this way a workflow for preparing a contrast agent-assisted imaging procedure is completed faster, since there is no need firstly to look up values in tables or to convert an injection protocol to determined values. It is furthermore likely in many cases that where patients are the examination subject, the determined weight is more consistent than the stated weight, since many patients are not quite correct when stating their weight, i.e. indicate too low a weight, for example.

The method according to an embodiment of the invention can be carried out particularly effectively if the at least one body dimension is determined automatically. An automated determination of the at least one body dimension speeds up the preparations for the contrast agent-assisted imaging and furthermore also permits inexperienced operating staff to perform the contrast agent-assisted imaging method.

Alternatively or in addition, landmarks of the examination subject can also be determined in an automated manner on the basis of the acquired external images and the at least one body dimension of the examination subject can be determined on the basis of at least one distance between the landmarks. The landmarks can for example mark positions of specific subregions of the examination subject and so supply additional details for the determination of relevant dimensions, which will then permit in turn a more exact determination of the cited protocol parameters. For example, the landmarks can mark the position of individual parts of a patient's body through which a contrast agent that is to be administered is intended to flow. The length of the transportation path through the body of the patient for the contrast agent can then be determined based on the knowledge of these positions. In practice, the feet or the head of a patient, for example, can be automatically identified as landmarks in the acquired images. The distance between the landmarks then yields the size of the person in camera image coordinates. Analogously thereto, the width of the person can also be determined in camera image coordinates by determining the distance between landmarks such as the shoulders, the knees or the left and right side of the hip of the person.

Heuristics can be used for the automated identification of the landmarks, which heuristics comprise at least one of the following methods:
the localization of landmarks with the aid of edge detectors,
a threshold filtering method,
a machine learning method.

Suitable features for localizing the landmarks can be identified automatically with the aid of the cited methods.

When edge detectors are used, differences in texture, in particular differences in contrast, in the acquired image are determined which point to a presence of demarcation lines between different objects or structures which can be used for the segmentation of an acquired image.

Threshold filters are also used for the segmentation of images. In this case the association between a pixel and a segment is determined by comparing a grayscale value or another feature with a threshold value. Owing to their simplicity, thresholding methods can be implemented quickly and segmentation results can be calculated with little overhead.

When a machine learning method is used, suitable features for localizing the landmarks are determined in an automated manner on the basis of annotated training images.

The cited methods serve for pattern recognition on the basis of the external image data acquired with the aid of the external image acquisition procedure. Landmarks typically feature characteristic structures which can be identified with the aid of the presented methods.

In a particularly preferred variant of the method according to an embodiment of the invention, the contours of the examination subject are determined on the basis of the externally acquired images and the dimensions of the examination subject are determined on the basis of the determined contours. For example, the volume of the examination subject can be determined more accurately on the basis of the contours of the examination subject. Furthermore, it may also be possible to distinguish individual different subregions of the examination subject in relation to which specific information is available which can be taken into account in the determination of the protocol parameters.

It is also advantageous if the distance of the image acquisition unit from the examination subject is taken into account in the determination of the at least one dimension and/or of the weight of the examination subject. In other words, the imaging scale of the external image acquisition is determined with knowledge of the distance of the examination subject from the external image acquisition unit. Further parameters to be taken into account may be for example the focal length of the lens of the external image acquisition unit. The actual dimensions of the examination subject can then be deduced from the cited parameters and the at least one dimension determined on the externally acquired image.

In a particularly advantageous variant of the method according to an embodiment of the invention, external images of the examination subject are acquired from different directions with the aid of the external image acquisition unit. The different directions may comprise a frontal view and a profile view, for example. The volume of the examination subject can be reconstructed on the basis of the external images acquired from several directions. Furthermore, flow paths of the contrast agent and their path length may also be determined more accurately, since in this case all three dimensions can be taken into consideration.

In order to determine the volume on the basis of a single acquired external image, methods such as the “shape from shading” technique may be used, for example. In this case, inferences are made on the basis of the lighting conditions in relation to the extent of a person also in the direction of the optical axis of the external image acquisition unit on the basis of a single externally acquired image. Within the scope of the present method, a reconstruction of a three-dimensional surface is carried out on the basis of the shadows cast in an externally acquired image.

Preferably the external image acquisition unit comprises at least one of the following devices:
a camera,
a depth-sensing camera,
a contactless electromagnetic sensor,
an ultrasonic distance meter,
a radar sensor device,
a depth-sensing camera and in addition a 2D camera.

The camera used may be for example a digital camera with which a two-dimensional image is taken in which specific anatomical features, the already mentioned landmarks, are identified. The camera may also be part of a smartphone or a tablet computer, for example.

In an acquisition of images with the aid of a depth-sensing camera, use is made of a camera which delivers a three-dimensional image, also referred to in the following as a 3D image. A depth-sensing camera generates an image in which each pixel indicates the distance of the nearest object from the camera. This information permits the depth image to be transformed into a point cloud in global coordinates. As in the 2D image, landmarks can be identified and distances determined in the 3D image. With a depth-sensing camera it is furthermore possible to determine the extent of the person along the optical axis and thus make a more accurate weight estimation.

Common methods used in the acquisition of a 3D image are the structured light method or the time-of-flight method. In the structured light method, line patterns are generated on the object that is to be imaged. These lines intersect on the object, for example. Due to the three-dimensional extension of the object, the intersecting lines are distorted, enabling a three-dimensional image of the object to be derived therefrom. With the time-of-flight method, a transit time measurement is taken of light beams that are emitted in the direction of an object that is to be imaged. Using a determined phase difference between emitted and received light waves as a basis, it is possible to deduce the distances present between the detection system used for the measurement and the object that is to be imaged.

The cited contactless electromagnetic sensors, ultrasonic distance meters or radar sensor devices may also be used in order to obtain a visualization of the patient in a 3D image.

A depth-sensing camera may additionally comprise a further 2D camera also. If both cameras are calibrated to one another, the determination of landmarks or contours can take the 2D image and the 3D image into account simultaneously, thereby improving the accuracy of the determination of dimensions and consequently the precision of the weight determination, since 2D cameras in most cases achieve a higher resolution than 3D cameras.

In images acquired via depth-sensing cameras or depth sensors, there is generally a lack of information in respect of the reverse side of the object that is to be imaged. If the object is located in a known environment, then this additional information relating to the environment can be used in order to improve the determination of the dimensions of the object. For example, a person can lie on a table the height of which is known. In this case the extent of the patient in an image acquired at right angles to the table can be determined as the distance between the table and the determined patient surface on the side facing away from the table.

In a special embodiment of the method according to an embodiment of the invention, a virtual model of the examination subject is used for particularly precise determination of the at least one body dimension of the examination subject, which virtual model is fitted to the data obtained via the external image acquisition procedure. If the examination subject is a human being, such a virtual model is commonly referred to as an avatar.

An avatar may be thought of as a kind of virtual jointed puppet which is inserted into the acquired external image data, in particular 3D image data, according to the patient's pose. An avatar may comprise a statistical form model which contains realistic proportions for the individual limbs and their dependencies from a database of images acquired from natural persons. If such an avatar is fitted into the acquired external image data, inaccuracies in the acquired external images, e.g. caused by noise or overexposure, can be compensated for. An avatar additionally provides information concerning the extension of the patient out of the image plane. By virtue of its structured hierarchical framework, the avatar permits the volume and the weight of the patient to be determined also for individual body regions and limbs.

It is particularly advantageous if the virtual model comprises personalized information in respect of the examination subject determined on the basis of a database, which information influences the start time of the contrast agent-assisted image acquisition procedure and/or the required contrast agent volume. To that end, relevant medical information such as image data, disease progressions, etc. is stored in a comprehensive database. For a patient that is to be examined, the person deemed most similar in terms of a suitable distance measure is then identified in the database.

Alternatively or in addition, a machine learning method, referred to as a deep learning method or a reinforcement learning method, may also be used for the synchronization with the database. In this case, on the one hand a body shape of the patient derived from the acquired external image data can be taken into account for example, and on the other hand a body shape can be used which is derived from the medical image data stored in the database. In addition, the patient's disease symptoms for example can also be taken into account and for example a search conducted in the database to find a patient with similar body shape and comparable parameters of the cardiovascular system. Once one or more similar patients have been located in the database, their relevant parameters, for example the weight or the rate of blood flow, are applied to the personalized avatar.

In addition, it is also possible to determine further influencing variables on the contrast agent diffusion with the aid of camera images. For example, gender recognition and/or age estimation can be carried out based on learning algorithms. A patient identification may also be performed on the basis of face recognition or the readout of a barcode armband. Moreover, an assessment in terms of the respiratory position of a patient may also be made with the aid of camera images. The cited variables are then also taken into account in the determination of the protocol parameters, with the result that the latter can be calculated with greater precision.

If a plurality of different contrast agents are used in a medical imaging method, the relevant parameters for the contrast agent application can be determined on a specific basis for each contrast agent. For example, different contrast agents may comprise different optimal contrast agent concentrations.

The use of the indefinite articles “a” or “an” does not exclude the possibility that the feature in question may also be present more than once. The use of the term “comprise” does not rule out the possibility that the concepts linked by way of the term “comprise” may be identical. For example, the imaging medical device comprises the imaging medical device. The use of the term “unit” does not rule out the possibility that the object to which the term “unit” refers may comprise a plurality of components that are separated from one another in space. The use of ordinal number terminology (first, second, third, etc.) in the designation of features serves in the context of the present application first and foremost to better differentiate the features designated using ordinal numbers. The absence of a feature which is designated by a combination of a given ordinal number and a term does not exclude the possibility that a feature may be present which is designated by a combination of an ordinal number following the given ordinal number and the term.

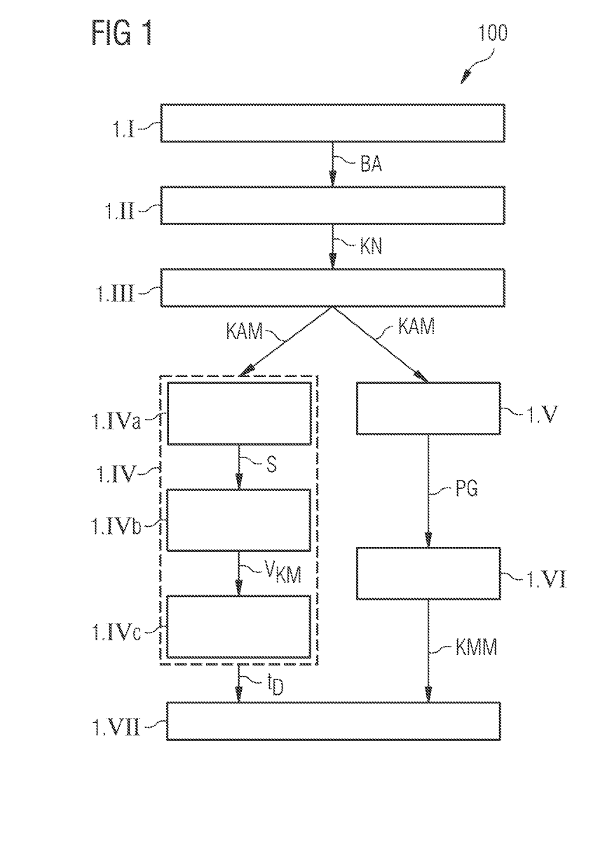

FIG. 1 shows a flowchart 100 by which an example embodiment of a method for determining at least one protocol parameter for a contrast agent-assisted acquisition of images of a region that is to be examined of an examination subject via a medical imaging device. Firstly, at step 1.1, external images BA of a patient are acquired with the aid of a camera. The camera is arranged in such a way relative to the examination subject that the contours KN of the patient can be recorded on the external images BA acquired with the aid of the camera. Next, at step 1.II, the contours KN of the patient are determined on the acquired external images BA. For this purpose, contrast differences in the acquired external images BA are taken into account, for example. Following this, at step 1.III, body dimensions KAM of the patient are determined on the basis of the acquired contours KN.

At step 1.IV, the body dimensions KAM are used to determine a start time t_{D} of a contrast agent-assisted image acquisition procedure. In the process, at step 1.Iva, a distance or path length s between an injection site for the administration of a contrast agent and a region of the patient that is to be examined is calculated on the basis of the body dimensions KAM. For this purpose, anatomical information from a database may also be used in addition in order for example to determine the position of the region to be examined in the body of the patient. Next, at step 1.IVb, a flow rate v_{KM} of a contrast agent is calculated on the basis of known injection parameters, such as, for example, the contrast agent volume injected per unit time and possibly the diameter of the arteries of the patient that are used for the transportation of the contrast agent. Finally, at step 1.IVc, the delay time t_{D} between the start of an administration of contrast agent and the start of the medical imaging is calculated or estimated from the quotient from the path length s and the flow rate v_{KM} of the contrast agent.

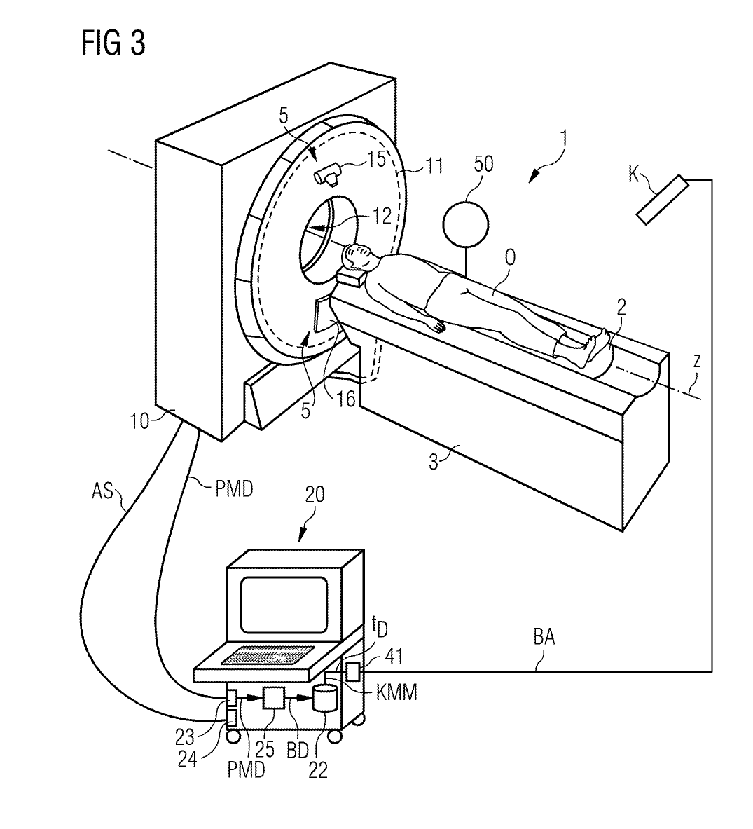

In addition, at step 1.V, the body dimensions KAM are used to calculate a weight PG of the patient O (see FIG. 3). The patient weight PG is then used at step 1.VI to determine a contrast agent volume KMM required for a subsequent medical imaging procedure. Finally, at step 1.VII, a medical imaging procedure is carried out on the basis of the values determined for the contrast agent volume KMM and the start time or, as the case may be, the delay time t_{D} between the injection of a contrast agent and the start of the medical imaging procedure.

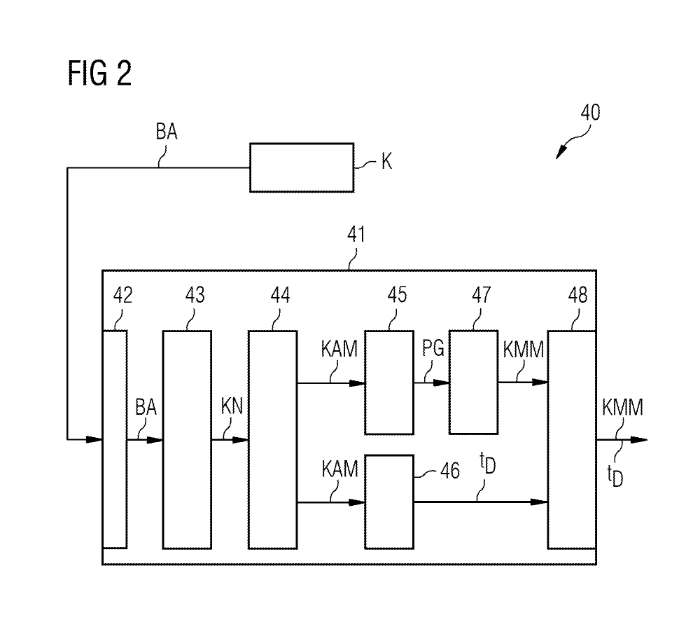

FIG. 2 shows an image acquisition parameter determination device 40 for determining at least one protocol parameter for a contrast agent-assisted acquisition of images of a region that is to be examined of an examination subject O (see FIG. 3) via a medical imaging device 1 (see FIG. 3). The image acquisition parameter determination device 40 comprises an image acquisition parameter determination unit 41 and in addition also a camera K by which external images BA of the examination subject can be acquired. The acquired external images BA recorded by the camera K are transmitted to the image acquisition parameter determination unit 41. The image acquisition parameter determination unit 41 comprises an input interface 42 by which the acquired external images BA are received. Subsequently, the acquired external image data BA is sent internally to a contour determination unit 43. The contour determination unit 43 determines contours KN of the body of the examination subject O on the basis of the acquired external image data BA.

Following this, the contour data KN is transmitted to a body dimension determination device 44. The body dimension determination device 44 determines body dimensions KAM of the examination subject O on the basis of the determined contours KN. The determined body dimension values KAM are then transmitted to a patient weight determination unit 45 and a start time determination unit 46. The patient weight determination unit 45 determines an individual patient weight PG on the basis of the body dimension values KAM. The determined patient weight value PG is subsequently used by a contrast agent volume determination unit 47 to calculate a suitable contrast agent volume KMM for the determined weight PG of the patient O, where necessary taking into account further parameters, such as physiological factors of the patient, for example. The start time determination unit 46 determines a start time t_{D} for the contrast agent-assisted image acquisition procedure on the basis of the body dimensions KAM of the patient O. Finally, the determined parameter values t_{D}, KMM are output via an output interface 48 to another unit such as a unit for determining a measurement protocol, for example.

FIG. 3 shows a computed tomography system 1 according to an example embodiment of the invention, which also comprises an image acquisition parameter determination unit 41 corresponding to the unit 41 shown in FIG. 2 according to an example embodiment. The CT system 1 in this case consists substantially of a conventional scan unit 10 in which a projection data acquisition unit 5 having a detector 16 and an X-ray source 15 positioned opposite the detector 16 and mounted on a gantry 11 revolves around a measurement chamber 12. Located in front of the scan unit 10 is a patient support device 3 or patient table 3, the upper part 2 of which can be maneuvered with a patient O positioned thereon toward the scan unit 10 in order to move the patient O through the measurement chamber 12 relative to the detector system 16. The scan unit 10 and the patient table 3 are controlled via a control device 20, from which there come, via a conventional control interface 24, acquisition control signals AS for the purpose of controlling the overall system in accordance with a predefined measurement protocol, taking into account the parameters t_{D}, KMM determined via the image acquisition parameter determination unit 41.

In the case of a spiral acquisition, a helical trajectory is produced as a result of a movement of the patient O along the z-direction, which corresponds to the system axis z lengthwise through the measurement chamber 12, and the simultaneous revolution of the X-ray source 15 for the X-ray source 15 relative to the patient O during the measurement. In parallel, the detector 16 constantly co-rotates in this case opposite the X-ray source 15 in order to acquire projection measurement data PMD, which is then used to reconstruct volume and/or slice image data.

Similarly, a sequential measurement method can also be performed in which a fixed position in the z-direction is approached and then the required projection measurement data PMD is acquired at the relevant z-position during one revolution, a partial revolution or several revolutions in order to reconstruct a slice image at the z-position or in order to reconstruct image data BD from the projection data of a plurality of z-positions. The inventive method 100 is basically also suitable for use on other CT systems, e.g. having a plurality of X-ray sources and/or detectors and/or having one detector forming a complete ring.

The projection measurement data PMD (also referred to in the following as raw data) acquired by the detector 16 in the course of a contrast agent-assisted imaging procedure is transferred to the control device 20 via a raw data interface 23. Following suitable preprocessing where appropriate (e.g. filtering and/or beam hardening correction), the raw data PMD is then processed further in an image reconstruction unit 25, which in the present example embodiment is realized in the form of software on a processor in the control device 20. The image reconstruction unit 25 reconstructs image data BD on the basis of the raw data PMD with the aid of a reconstruction method. A reconstruction method based on filtered back-projection may be used as the reconstruction method, for example.

The acquired image data BD is stored in a memory 22 of the control device 20 and/or output in the usual way on the screen of the control device 20. The data can also be fed via an interface not shown in FIG. 3 into a network connected to the computed tomography system 1, for example a radiological information system (RIS), and stored in a mass storage facility that is accessible there or output as images on printers or filming stations connected there. The data can thus be processed further in any desired manner and then stored or output.

In addition, FIG. 3 also shows an image acquisition parameter determination unit 41, which receives external image data BA of the patient O from a camera K. On the basis of the external image data BA, the image acquisition parameter determination unit 41 determines, as described in connection with FIGS. 1 and 2, protocol parameters t_{D}, KMM for an image acquisition protocol of the CT system 1. The image acquisition parameter determination unit 41 is depicted in FIG. 3 as part of the control device 20. The determined protocol parameters t_{D}, KMM can be stored for example in the memory device 22 and used for a later CT imaging procedure by the CT system 1. Furthermore, the CT system shown in FIG. 3 also comprises a contrast agent injection device 50, by which the patient O may be injected prior to the commencement of a CT imaging method with a contrast agent whose behavior in a vessel or a vascular system, for example, is captured in the form of images with the aid of the computed tomography system 1.

In conclusion, it is pointed out once again that the above-described method for determining at least one protocol parameter for a contrast agent-assisted acquisition of images and the described image acquisition parameter determination device 41 as well as the described computed tomography system 1 are simply preferred example embodiments of the invention and that the invention may be varied by the person skilled in the art without departing from the scope of the invention as defined by the claims. For example, a magnetic resonance tomography system may also be used as an imaging system. It is also pointed out for the sake of completeness that the use of the indefinite articles “a” or “an” does not exclude the possibility that the features in question may also be present more than once. Similarly, the term “unit” does not rule out the possibility that this consists of a plurality of components which, where necessary, may also be distributed in space.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for determining at least one protocol parameter for a contrast agent-assisted acquisition of images of a region that is to be examined of an examination subject via a medical imaging device, the method comprising:
acquiring external images of externally visible features of the examination subject with the aid of an external image acquisition unit;
determining at least one body dimension of the examination subject on the basis of the acquired external images; and
determining at least one contrast agent protocol parameter on the basis of the at least one determined body dimension.

2. The method of claim 1, wherein the at least one determined contrast agent protocol parameter comprises one of the following variables:
a required contrast agent volume, and
a start time of the contrast agent-assisted image acquisition procedure.

3. The method of claim 2, further comprising:
determining a weight of the examination subject on the basis of the at least one body dimension determined on the basis of the externally acquired images; and
determining the required contrast agent volume on the basis of the determined weight of the examination subject.

4. The method of claim 1, wherein at least one of
the at least one body dimension is determined in an automated manner and
landmarks of the examination subject are determined in an automated manner on the basis of the acquired external images, wherein the at least one body dimension of the examination subject is determined on the basis of at least one distance between the landmarks.

5. The method of claim 4, wherein at least one of the following methods is used for the automated determination of the landmarks:
an edge detection method,
a threshold filtering method, and
a machine learning method.

6. The method of claim 1, wherein contours of the examination subject are determined based on the acquired external images and the dimensions of the examination subject are determined on the basis of the determined contours.

7. The method of claim 3, wherein a distance of the external image acquisition unit from the examination subject is taken into account in the determination of at least one of the at least one body dimension and the weight of the examination subject.

8. The method of claim 1, wherein external images of the examination subject are acquired from different directions with the aid of the external image acquisition unit.

9. The method of claim 1, wherein the external image acquisition unit comprises at least one of the following devices:
a camera,
a depth-sensing camera,
a contactless electromagnetic sensor,
an ultrasonic distance metering unit,
a radar sensor device, and
a depth-sensing camera and in addition a 2D camera.

10. The method of claim 1, wherein a virtual model of the examination subject is used in order to determine the at least one body dimension of the examination subject, the virtual model being fitted to the data of the acquired external images.

11. The method of claim 10, wherein the virtual model comprises personalized information in respect of the examination subject determined on the basis of a database, the personalized information influencing at least one of a start time of the contrast agent-assisted image acquisition procedure and the required contrast agent volume.

12. An image acquisition parameter determination device for determining at least one protocol parameter for a contrast agent-assisted acquisition of images of a region that is to be examined of an examination subject via a medical imaging device, the image acquisition parameter determination device comprising:
an external image acquisition unit to acquire external images of externally visible features of the examination subject;
a body dimension determination device to determine at least one body dimension of the examination subject on the basis of the acquired external images; and
a contrast agent protocol parameter determination unit to determine at least one contrast agent protocol parameter on the basis of the at least one determined body dimension.

13. An imaging medical device, comprising:
a scan unit to scan a region that is to be examined of an examination subject;
a control unit to control the scan unit; and
the image acquisition parameter determination device of claim 12.

14. A non-transitory computer program product including a computer program, directly loadable into a memory device of a control device of an imaging medical device, including program sections for carrying out the method of claim 1 when the computer program is executed in the control device of the imaging medical device.

15. A non-transitory computer-readable medium storing program sections, readable in and executable by a computer unit, to carry out the method of claim 1 when the program sections are executed by the computer unit.

16. The method of claim 2, wherein at least one of
the at least one body dimension is determined in an automated manner and
landmarks of the examination subject are determined in an automated manner on the basis of the acquired external images, wherein the at least one body dimension of the examination subject is determined on the basis of at least one distance between the landmarks.

17. The method of claim 16, wherein at least one of the following methods is used for the automated determination of the landmarks:
an edge detection method,
a threshold filtering method, and
a machine learning method.

18. The method of claim 1, wherein a distance of the external image acquisition unit from the examination subject is taken into account in the determination of the at least one body dimension.

19. The method of claim 2, wherein external images of the examination subject are acquired from different directions with the aid of the external image acquisition unit.

20. The method of claim 2, wherein the external image acquisition unit comprises at least one of the following devices:
a camera,
a depth-sensing camera,
a contactless electromagnetic sensor,
an ultrasonic distance metering unit,
a radar sensor device, and
a depth-sensing camera and in addition a 2D camera.

21. The method of claim 2, wherein a virtual model of the examination subject is used in order to determine the at least one body dimension of the examination subject, the virtual model being fitted to the data of the acquired external images.

22. The method of claim 21, wherein the virtual model comprises personalized information in respect of the examination subject determined on the basis of a database, the personalized information influencing at least one of a start time of the contrast agent-assisted image acquisition procedure and the required contrast agent volume.

23. The imaging medical device of claim 13, wherein the imaging medical device is a computed tomography system.
